(19) United States
(12) Patent Application Publication
(10) Pub. No.: US 2022/0330795 A1
(43) Pub. Date: Oct. 20, 2022

(54) OMNI-DIRECTIONAL ENDOSCOPE MECHANISM

(71) Applicant: Shenzhen Avanline Digital-tech Co.,Ltd, Shenzhen (CN)

(72) Inventors: Zhigang Zeng, Qingyuan City (CN); Gangwen Zhang, Xi'an City (CN)

(21) Appl. No.: 17/659,252

(22) Filed: Apr. 14, 2022

(30) Foreign Application Priority Data
Apr. 16, 2021 (CN) 202120775251.2

(51) Int. Cl. A61B 1/00

(52) U.S. Cl. CPC A61B 1/00121; A61B 1/00042; A61B 1/00045; A61B 2560/0487

(57) ABSTRACT

An omni-directional endoscope mechanism, one-handed joystick drives steel cable traction frame to swing and tilt, pulls steel cables and achieves bending part at front bent.

### CROSS-REFERENCE TO RELATED APPLICATIONS

This is a U.S. patent application which claims the priority and benefit of Chinese Patent Application Number 202120775251.2, filed on Apr. 16, 2021, the disclosure of which is incorporated herein by reference in its entirety.

### FIELD OF THE INVENTION

The utility model relates to the technical field of medical devices, in particular to an omni-directional endoscope mechanism.

### BACKGROUND ART

Endoscope is a testing instrument integrating traditional optics, ergonomics, precision machinery, modern electronics, mathematics and software. It has image sensors, optical lens, light source illumination, mechanical devices, etc. Its front-end working part can enter narrow spaces or human body, and can show the internal defects of products or human body lesions, which is very useful for engineers or doctors.

The existing endoscopes need two hands to operate at the same time, which results in that the users can't perform other operations at the same time, and the user experience is low.

### SUMMARY OF THE INVENTION

The purpose of the utility model is to provide an omni-directional endoscope mechanism which can be operated with one hand to solve the problem that the existing endoscopes need to be operated with both hands.

The utility model provides an omni-directional operating endoscope mechanism, which comprises a housing, a display screen arranged at one side of the housing, an operating mechanism arranged in the housing, four steel cables connected with the operating mechanism, and a bending part connected with the steel cables. The operating mechanism comprises an joystick, a hemispherical steel cable traction frame connected with the joystick, a universal rotating pair arranged at the lower end of the steel cable traction frame, a middle plate arranged at the lower end of the universal rotating pair, a retaining shell arranged at the lower end of the middle plate, a steel cable holder arranged at the lower end of the retaining shell, four rollers arranged in the holding shel, four steel cable connectors arranged around the steel cable traction frame, four through holes arranged in the middle of the middle plate, four steel cables arranged with one end of each steel cable connected with one of the four steel cable connectors, and the other end connected with the bending part after passing through the through hole and the steel cable holder in sequence.

When the user operates the joystick, the omni-directional operation endoscope mechanism drives the steel cable traction frame to swing around the universal rotating pair and to tilt, and the steel cables connected with the steel cable connectors are pulled to move, so that the bending part at the front end bends. In this process, only the joystick needs to be operated, so one-hand operation can be realized.

Furthermore, 4 guide tubes are arranged in the middle of the steel cable holder at positions corresponding to the 4 steel cables.

Furthermore, a locking mechanism is arranged on the side of the housing where the steel cable traction frame is located.

Further, the universal rotating pair comprises an upper fork, a lower fork and a cross hole shaft connecting the upper fork and the lower fork, and the joystick is connected with the steel cable traction frame and the upper fork.

Further, the locking mechanism comprises a locking key, a pressure plate arranged at the lower end of the locking key, a guide post connected with the pressure plate, and a return spring arranged between the guide post and the pressure plate, wherein the locking key is slidably connected with the housing, and the pressure plate is arranged around the outside of the universal rotating pair and the hemispherical steel cable traction frame at the upper part thereof, with one end of the pressure plate rotatably connected with the housing, and the other end arranged at the lower end of the locking key, a locking protrusion is arranged on the pressure plate at the position corresponding to the locking key, and a latch buckle is arranged on the locking key at the position corresponding to the locking protrusion.

Further, the latch buckle comprises a connecting part and a hook-shaped head arranged at the tail end of the connecting part, and a barb is arranged on the side of the upper end of the locking protrusion close to the locking key.

### BRIEF DESCRIPTION OF THE DRAWINGS

FIG. 1 is a schematic diagram of the three-dimensional structure of the omni- directional endoscope mechanism in the first embodiment of the utility model;

FIG. 2 is a schematic diagram of the first internal structure of the manipulation mechanism in the omni-directional endoscope mechanism of FIG. 1;

FIG. 3 is a schematic diagram of the second internal structure of the manipulation mechanism in the omni-directional endoscope mechanism of FIG. 1;

FIG. 4 is a schematic diagram of the three-dimensional structure of the universal rotating pair in the omni-directional endoscope mechanism of FIG. 1;

FIG. 5 is a schematic diagram of transverse section structure of the locking mechanism in the omni-directional endoscope mechanism in the second embodiment of the utility model;

FIG. 6 is a schematic diagram of the longitudinal section structure of the locking mechanism in the omni-directional endoscope mechanism of FIG. 5;

FIG. 7 is an enlarged schematic diagram of structure of the locking mechanism in the omni-directional endoscope mechanism of FIG. 6.

Description of main component symbols:

| | | | | | |
|---|---|---|---|---|---|
| Housing | 10 | The second | 335 | Locking key | 81 |
| | | rotating shaft | | | |
| Display screen | 20 | Middle plate | 34 | Latch buckle | 811 |
| Operating | 30 | Through- | 341 | Interconnecting | 8111 |
| mechanism | | hole | | piece | |
| Joystick | 31 | Retaining | 35 | Hook-shaped | 8112 |
| | | shell | | head | |
| Steel cable | 32 | Cable holder | 36 | Pressure plate | 82 |
| traction frame | | | | | |
| Steel cable | 321 | Roller | 37 | Locking | 821 |
| connector | | | | protrusion | |
| Universal | 33 | Steel cable | 40 | Barb | 8211 |
| rotating pair | | | | | |
| Upper fork | 331 | Bending part | 50 | Guide post | 83 |
| Lower fork | 332 | Guide tube | 60 | Return spring | 84 |
| Cross hole | 333 | Handle Bar | 70 | | |
| shaft | | | | | |
| The first | 334 | Locking | 80 | | |
| rotating shaft | | mechanism | | | |

The following detailed description will further explain the utility model with reference to the above drawings.

### DETAILED DESCRIPTION OF THE PREFERRED EMBODIMENTS

For the convenience of understanding the utility model, the utility model will be described more comprehensively with reference to related drawings. Several examples of the utility model are given in the attached drawings. However, the utility model can be realized in many different forms, and is not limited to the embodiments described herein. On the contrary, the purpose of providing these examples is to make the disclosure of the utility model more thorough and comprehensive.

It should be noted that when an element is refered to as “fixed” to another element, it may be directly on another element or there may be an intermediate element. When an element is considered to be “connected” to another element, it may be directly connected to another element or may have intermediate elements at the same time as another element. The terms “vertical”, “horizontal”, “left”, “right” and similar expressions used in this document are for illustrative purposes only.

Unless otherwise defined, all technical and scientific terms used in this document have the same meanings as those commonly understood by the technicians in the technical field of this utility model. The terms used in the specification of the utility model in this document are only for the purpose of describing specific embodiments, and are not intended to limit the utility model. As used herein, the term “and/or” includes any and all combinations of one or more related listed items.

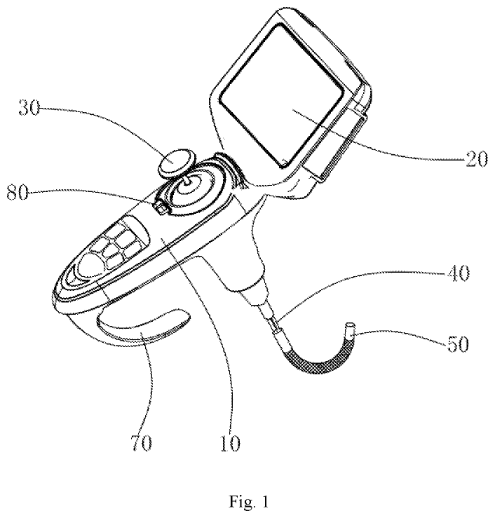

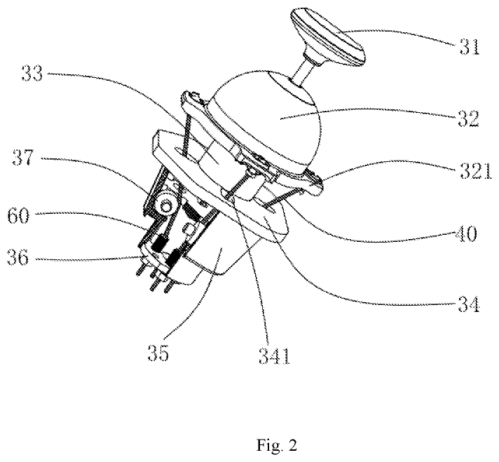

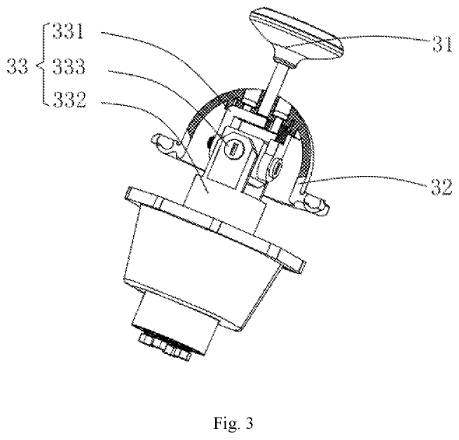

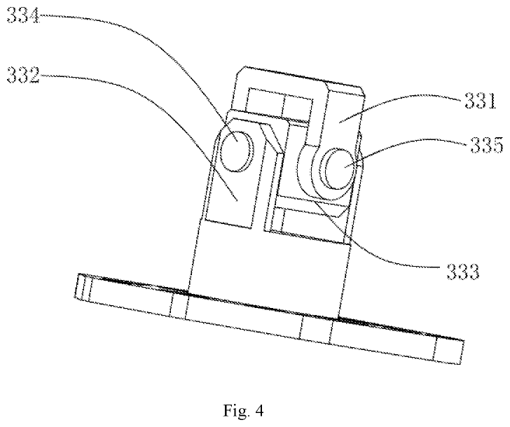

Referring to FIG. 1 to FIG. 4, the first embodiment of the utility model provides an omni-directional endoscope mechanism, which includes a housing 10, a display screen 20 arranged at one side of the housing 10, an operating mechanism 30 arranged in the housing 10, 4 steel cables 40 connected with the operating mechanism 30, and a bending part 50 connected with the steel cables 40.

Specifically, in this embodiment, the operating mechanism 30 includes a joystick 31, a hemispherical steel cable traction frame 32 connected to the joystick 31, a universal rotating pair 33 arranged at the lower end of the steel cable traction frame 32, a middle plate 34 arranged at the lower end of the universal rotating pair 33, a retaining shell 35 arranged at the lower end of the middle plate 34, a steel cable holder 36 arranged at the lower end of the retaining shell 35, and four rollers 37 arranged in the retaining shell 35. Four cable connectors 321 are arranged around the cable traction frame 32, four through holes 341 are arranged in the middle of the middle plate 34, and four cables 40 are arranged, with one end of each cable 40 connected with one of the cable connectors 321, the other end connected with the bending part 50 after passing through one of the through holes 341 and the cable holder 36 in sequence.

When the user operates the joystick 31, the above-mentioned omni-directional endoscope mechanism drives the cable traction frame 32 to swing around the universal rotation pair 33 to tilt, and then the cables 40 connected with the cable connectors 321 are pulled to move, so that the bending part 50 at the front end bends. In this process, only the joystick 31 needs to be operated, and the joystick will not be limited to swing in some planes due to the action of the universal rotation pair 33, thus realizing the omni-directional bending control of the bending part.

Specifically, in this embodiment, four guide tubes 60 are provided at the middle of the steel cable holder 36 at the positions corresponding to the steel cables 40 to guide the steel cables 40, and four rollers 37 further guide the steel cables and make the steel cables move smoothly.

Specifically, in this embodiment, the universal rotating pair 33 includes an upper fork 331, a lower fork 332 and a cross hole shaft 333 connecting the upper fork 331 and the lower fork 332. The cross hole shaft 333 is provided with a cross hole and is correspondingly provided with a first rotating shaft 334 and a second rotating shaft 335. The upper fork 331 can swing around the second rotating shaft 335, and the cross-hole shaft can swing around the first rotating shaft 334. The joystick 31 is connected with the cable traction frame 32 and the upper fork 331. Through the above structural design, the universal rotating pair 33 can realize the universal rotating function.

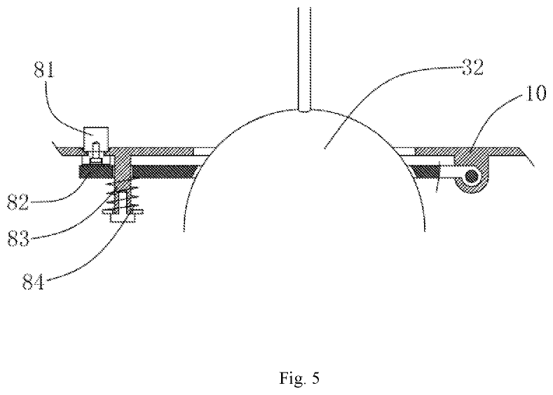

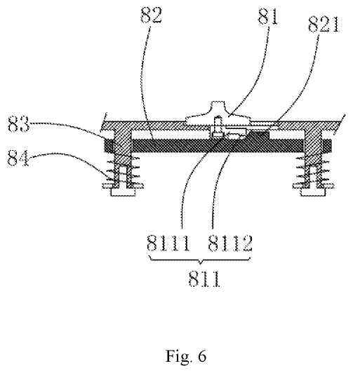

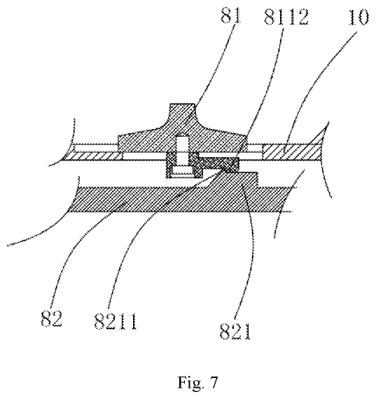

Please refer to FIG. 5 to FIG. 7. The second embodiment of the utility model provides an omni-directional endoscope mechanism. The difference between the second embodiment and the first embodiment is that in the second embodiment, the housing 10 is provided with a locking mechanism 80 at one side of the cable traction frame 32. Specifically, in this embodiment, the locking mechanism 80 includes a locking key 81, a pressing plate 82 arranged at the lower end of the locking key 81, a guide post 83 connected with the pressing plate 82, and a return spring 84 arranged between the guide post 83 and the pressing plate 82, wherein the locking key 81 is slidably connected with the housing 10, the pressing plate 82 is arranged around the outside the universal rotating pair 33 and the hemispherical steel cable traction frame at its upper part, and one end of the pressing plate 82 is rotatably connected with the housing 10, while the other end is arranged at the lower end of the locking key 81. The pressing plate 82 is provided with a locking protrusion 821 at a position corresponding to the locking key 81, and the locking key 81 is provided with a latch buckle 811 at a position corresponding to the locking protrusion 821. When the locking key 81 is pushed forward, the latch buckle 811 presses down the locking protrusion 821, so that the pressing plate 82 presses the steel cable traction frame 32, and the universal rotating pair 3 can't move, thus playing a locking role. When the locking key 81 is pushed back, the latch buckle 811 and the locking protrusion 821 are disengaged, and under the action of the return spring, the end of the pressure plate that was pressed against the steel cable traction frame is lifted, and the joystick is restored to the movable state.

Specifically, in this embodiment, the latch buckle 811 includes a connecting part 8111 and a hook-shaped head 8112 arranged at the end of the connecting part 8111, and a barb 8211 is arranged at the side of the upper end of the locking protrusion 821 near the locking key 81. Through the above structural design, the locking key 81 can keep the locked state.

The above-mentioned examples only express several embodiments of the utility model, and their descriptions are more specific and detailed, but they can't be understood as limiting the patent scope of the utility model. It should be pointed out that, for ordinary technicians in this field, under the premise of not deviating from the concept of this utility model, several changes and improvements can be made, which all belong to the protection scope of this utility model. Therefore, the scope of protection of this utility model patent shall be subject to the appended claims.

What is claimed is:

1. An omni-directional endoscope mechanism comprises a housing, a display screen arranged at one side of the housing, an operating mechanism arranged in the housing, four steel cables connected with the operating mechanism, and a bending part connected with the steel cables. It is characterized in that, the operating mechanism comprises a joystick, a hemispherical steel cable traction frame connected with the joystick, a universal rotating pair arranged at the lower end of the steel cable traction frame, a middle plate arranged at the lower end of the universal rotating pair, a retaining shell arranged at the lower end of the middle plate, a steel cable holder arranged at the lower end of the retaining shell, and four rollers arranged in the holding shell, four steel cable connectors arranged around the steel cable traction frame, four through holes arranged in the middle of the middle plate, four steel cables arranged with one end of each steel cable connected with one of the steel cable connectors, and the other end connected with the bending part after passing through the through hole and the steel cable holder in sequence.

2. The omni-directional endoscope mechanism according to claim 1, characterized in that 4 guide tubes are arranged at the position corresponding to the steel cables in the middle of the steel cable holder.

3. The omni-directional operation endoscope mechanism according to claim 1, characterized in that the side of the shell with the steel cable traction frame is provided with a locking mechanism.

4. The omni-directional endoscope mechanism according to claim 1, characterized in that the universal rotating pair comprises an upper fork, a lower fork and a cross hole shaft connecting the upper fork and the lower fork, and the joystick is connected with the steel cable traction frame and the upper fork.

5. The omni-directional endoscope mechanism according to claim 3, characterized in that the locking mechanism comprises a locking key, a pressure plate arranged at the lower end of the locking key, a guide post connected with the pressure plate, and a return spring arranged between the guide post and the pressure plate, wherein the locking key is slidably connected with the housing, the pressure plate is arranged arround the outside of the universal rotating pair and the hemispherical steel cable traction frame at the upper part thereof, with one end of the pressure plate rotatably connected with the housing, and the other end arranged at the lower end of the locking key, a locking protrusion is arranged on the pressure plate at a position corresponding to the locking key, and a latch buckle is arranged on the locking key at the position corresponding to the locking protrusion.

6. The omni-directional endoscope mechanism according to claim 5, characterized in that the latch buckle comprises a connecting part and a hook-shaped head arranged at the end of the connecting part, and a barb is arranged on the side of the upper end of the locking protrusion close to the locking key.
